SEQUENCE LISTING (10 sequences)

SEQ ID NO: 1 - protein, 5 aa, synthetic construct
AYSVN

SEQ ID NO: 2 - protein, 16 aa, synthetic construct
MIWGDGKIVYNSALKS

SEQ ID NO: 3 - protein, 10 aa, synthetic construct
DGYYPYAMDN

SEQ ID NO: 4 - protein, 15 aa, synthetic construct
RASKSVDSYGNSFMH

SEQ ID NO: 5 - protein, 7 aa, synthetic construct
LASNLES

SEQ ID NO: 6 - protein, 9 aa, synthetic construct
QQNNEDPRT

SEQ ID NO: 7 - protein, 117 aa, synthetic construct
VTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 8 - protein, 111 aa, synthetic construct [sequence omitted]

SEQ ID NO: 9 - protein, 445 aa, synthetic construct
  VARIANT 1: note = X at position 1 is Q, pyro-glutamic acid, or absent
  VARIANT 444: note = X at position 444 is G or absent
  VARIANT 445: note = X at position 445 is K or absent
XVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLXX

SEQ ID NO: 10 - protein, 218 aa, synthetic construct
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0242456 A1
(43) Pub. Date: Aug. 20, 2026

(54) IL-13 ANTIBODIES FOR THE TREATMENT OF ATOPIC DERMATITIS

(71) Applicant: DERMIRA, INC., Indianapolis, IN (US)

(72) Inventors: Amber ATWATER, Great Falls, VA (US); Eric R. MESKIMEN, Plainfield, IN (US); Sreekumar PILLAI, Carmel, IN (US); Maria Jose RUEDA, Boston, MA (US)

(21) Appl. No.: 19/162,763

(22) PCT Filed: Mar. 5, 2024
(86) PCT No.: PCT/US2024/018484

§ 371 Date: Sep. 5, 2025

Related U.S. Application Data
(60) Provisional application No. 63/489,029, filed on Mar. 8, 2023.

(51) Int. Cl. C07K 16/24; A61K 31/573; A61K 31/58; A61K 39/00; A61K 39/395; A61P 17/00

(52) U.S. Cl. CPC C07K 16/244; A61K 31/573; A61K 31/58; A61K 39/3955; A61P 17/00; A61K 2039/505; C07K 2317/24

(57) ABSTRACT

Provided herein are methods, uses and pharmaceutical compositions of antibodies that bind human IL-13 (“anti-IL-13 antibodies”) for treating atopic dermatitis, e.g., in a patient who has been previously administered dupilumab.

### SEQUENCE LISTING

The present application is being filed along with a Sequence Listing in ST.26 XML format. The Sequence Listing is provided as a file titled “30602_WO Sequence Listing ST26” created Feb. 8, 2024, and is 12 kilobytes in size. The Sequence Listing information in the ST.26 XML format is incorporated herein by reference in its entirety.

### FIELD

The present invention relates to methods, uses and pharmaceutical compositions of antibodies that bind human IL-13 (“anti-IL-13 antibodies”) for treating atopic dermatitis, e.g., in a patient who has been previously administered dupilumab.

### BACKGROUND

Atopic dermatitis (AD) is a chronic heterogeneous inflammatory skin disorder caused by skin barrier dysfunction and dysregulation of the immune response. Clinically, AD is characterized by xerosis, erythematous crusting rash, lichenification, an impaired skin barrier, and intense pruritus (Bieber T., N Engl J Med 2008; 358:1483-94). Patients with AD have a high disease burden, and their quality of life is significantly impacted. In one study, AD was shown to have a greater negative effect on patient mental health than diabetes and hypertension (Zuberbier T, et al., J Allergy Clin Immunol 2006; 118:226-32). Patients with moderate to severe AD have a higher prevalence of social dysfunction and sleep impairment, which is directly related to severity of disease (Williams H, et al., J Allergy Clin Immunol 2008; 121:947-54.e15). Depression, anxiety, and social dysfunction not only affect patients with AD but also their caregivers (Zuberbier T, et al., J Allergy Clin Immunol 2006; 118:226-32).

Interleukin (IL)-13 is a key mediator of T-helper type 2 (Th2) inflammation and signals through a heterodimeric receptor IL-4Ra/lL-13Ra1. Several lines of evidence suggest that IL-13 is a key pathogenetic component in AD. Increased expression of IL-13 has consistently been reported in AD skin (Hamid Q, et al., J Allergy Clin Immunol 98:225-31 [1996]; Jeong C W, et al., Clin Exp Allergy 33:1717-24 [2003]; Tazawa T, et al., Arch Dermatol Res 295:459-64 [2004]; Neis M M, et al., J Allergy Clin Immunol 118:930-7 [2006]; Suárez-Fariñas M, et al., J Allergy Clin Immunol 132:361-70 [2013]; Choy D F, et al., J Allergy Clin Immunol. 130:1335-43 [2012]) and some reports suggest a relationship between IL-13 expression and the severity of disease (La Grutta S, et al., Allergy 60:391-5 [2005]). Increased IL-13 has also been reported in the serum of AD patients (Novak N, et al., J Invest Dermatol 2002; 119:870-5; WO2016149276), and several studies have reported an increase in IL-13-expressing T cells in the blood of AD patients (Akdis M, et al., J Immunol 1997; 159:4611-9; Aleksza M, et al., Br J Dermatol 2002; 147:1135-41; La Grutta S, et al., Allergy 2005; 60:391-5).

Due to the heterogeneous nature of the disease, patients have diverse treatment needs. The therapeutic approaches to AD include trigger avoidance, skin hydration with bathing and use of emollients and anti-inflammatory therapies such as topical corticosteroids (TCS). In many patients, treatment with TCS provides some measure of symptomatic relief but does not adequately control their disease. In addition, TCS use is associated with many comorbidities and limitations including high patient burden. Long-term application of TCS is not recommended because of the risk of skin atrophy, dyspigmentation, acneiform eruptions, and risks associated with systemic absorption (e.g., hypothalamic pituitary axis effects, Cushing's disease).

For patients who have persistent moderate to severe AD and do not respond adequately to TCS, there are a number of step-up therapeutic options (Ring J, et al., J Eur Acad Dermatol Venereol 2012; 26:1176-93; Schneider L, et. al., J Allergy Clin Immunol 2013; 131:295-9. e1-27). The step-up options include topical calcineurin inhibitors (TCI), phototherapy, and immunosuppressive agents such as oral corticosteroids, cyclosporine, azathioprine, methotrexate, and mycophenolate. Immunosuppressants like cyclosporine A affect both humoral and cellular immune responses, which could lead to increased susceptibility to infections and decreased cancer immunosurveillance.

Dupilumab, a monoclonal antibody which inhibits the signaling of IL-4 and IL-13, was approved for the treatment of patients with moderate-to-severe AD. Dupilumab was the first available biologic for patients with chronic moderate-to-severe AD for which topical treatment provided inadequate control or was medically inadvisable. A proportion of patients that undergo treatment with dupilumab may show a limited or partial response or even no response at all to treatment. Other patients may show an initial response to treatment but lose response over time. Still others are found to be intolerant or have adverse effects to dupilumab. Additionally, in real world practice other patients discontinue treatment due to issues such as cost or loss of access (for example, insurance) to the drug. Taken together, a significant number of patients do not continue dupilumab treatment over the course of their chronic disease (Simpson et al. 2016, N Eng/J Med., 375:2335-2348; Marniquet et al. 2022, Br J Dermatol. 186: 733-734; Faiz et al. 2019, J Am Acad Dermatol., 81:143-51).

There remains an unmet medical need for safer and more effective therapies and treatment regimens for moderate to severe AD, especially for patients who has been previously administered dupilumab.

### SUMMARY OF INVENTION

Provided herein are methods, uses and pharmaceutical compositions of anti-IL-13 antibodies for treating atopic dermatitis. In some embodiments, provided herein are methods and uses of anti-IL-13 antibodies for treating atopic dermatitis, e.g., in a patient who has been previously administered dupilumab.

In one aspect, provided herein are methods of treating moderate to severe atopic dermatitis in a patient in need thereof who has been previously administered dupilumab, which comprise administering to the patient an antibody that binds human IL-13. In some embodiments, provided herein are methods for treating moderate to severe atopic dermatitis, which comprise selecting a patient who has been previously administered dupilumab, and administering to the patient an antibody that binds human IL-13. In some embodiments, the patients stopped dupilumab due to no response, partial response, or loss of response. In some embodiments, the patients stopped dupilumab due to intolerance or adverse events. In some embodiments, the patients stopped dupilumab for other reasons.

In some embodiments, provided herein are methods for treating moderate to severe atopic dermatitis, which comprise selecting a patient who has moderate to severe atopic dermatitis and has been previously administered dupilumab and stopped dupilumab due to non-response, partial response, or loss of response, and administering to the patient an antibody that binds human IL-13.

In some embodiments, provided herein are methods for treating moderate to severe atopic dermatitis, which comprise selecting a patient who has moderate to severe atopic dermatitis and has been previously administered dupilumab and stopped dupilumab due to intolerance or adverse event, and administering to the patient an antibody that binds human IL-13.

In some embodiments, the patient has moderate to severe atopic dermatitis for at least a year. In some embodiments, the moderate to severe atopic dermatitis is determined by the American Academy of Dermatology Consensus Criteria. In some embodiments, the patient has an Eczema Area and Severity Index (EASI) score of 16 or greater, an Investigator Global Assessment (IGA) score of 3 or greater, and more than 10% of body surface area (BSA) affected by atopic dermatitis, before administration of the antibody. In some embodiments, the patient had inadequate response to topical corticosteroids. In some embodiments, the patient is 12 years of age or older.

Also provided herein are methods for treating moderate to severe atopic dermatitis, which comprise selecting a patient who (i) is 12 years of age or older; (ii) has chronic atopic dermatitis according to American Academy of Dermatology Consensus Criteria for more than a year; (iii) has an EASI score of 16 or greater; (iv) has an IGA score of 3 or greater; (v) has more than 10% of body surface area affected by atopic dermatitis; (vi) had inadequate response to topical corticosteroids; and (vii) has been previously administered dupilumab, wherein the patient stopped dupilumab due to (1) non-response, partial response, or loss of response, or (2) intolerance or adverse event; and administering to the patient an antibody that binds human IL-13.

In another aspect, provided herein is an antibody that binds human IL-13 for use in the treatment of moderate to severe atopic dermatitis in a patient who has been previously administered dupilumab. In some embodiments, provided herein is an antibody that binds human IL-13 for use in the treatment of moderate to severe atopic dermatitis in a patient who has been previously administered dupilumab and stopped dupilumab due to non-response, partial response, or loss of response. In some embodiments, provided herein is an antibody that binds human IL-13 for use in the treatment of moderate to severe atopic dermatitis in a patient who has been previously administered dupilumab and stopped dupilumab due to intolerance or adverse event. In some embodiments, the patient: (1) is 12 years of age or older; (2) has chronic atopic dermatitis according to American Academy of Dermatology Consensus Criteria for more than a year; (3) has an EASI score of 16 or greater; (4) has an IGA score of 3 or greater; (5) has more than 10% of BSA affected by atopic dermatitis; and (6) had inadequate response to topical corticosteroids.

In another aspect, provided herein are uses of an antibody that binds human IL-13 in the manufacture of a medicament for the treatment of moderate to severe atopic dermatitis in a patient who has been previously administered dupilumab. Also provided herein are uses of an antibody that binds human IL-13 in the manufacture of a medicament for the treatment of moderate to severe atopic dermatitis in a patient who has been previously administered dupilumab and stopped dupilumab due to non-response, partial response, or loss of response. Also provided herein are uses of an antibody that binds human IL-13 in the manufacture of a medicament for the treatment of moderate to severe atopic dermatitis in a patient who has been previously administered dupilumab and stopped dupilumab due to intolerance or adverse event. In some embodiments, the patient: (1) is 12 years of age or older; (2) has chronic atopic dermatitis according to American Academy of Dermatology Consensus Criteria for more than a year; (3) has an EASI score of 16 or greater; (4) has an IGA score of 3 or greater; (5) has more than 10% of BSA affected by atopic dermatitis; and (6) had inadequate response to topical corticosteroids.

In some embodiments, the patient stopped dupilumab due to non-response, partial response, or loss of response. In some embodiments, the patient stopped dupilumab due to intolerance or adverse event. In some embodiments, the patient stopped dupilumab due to non-response. In some embodiments, the patient stopped dupilumab due to partial response. In some embodiments, the patient stopped dupilumab due to loss of response. In some embodiments, the patient stopped dupilumab for at least four weeks. In some embodiments, the patient stopped dupilumab for at least eight weeks.

In some embodiments, the antibody binds human IL-13 with high affinity and blocks signaling through the active IL-4Ralpha/IL-13Ralpha1 heterodimer. In some embodiments, the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the antibody that binds human IL-13 comprises a VH comprising SEQ ID NO: 7, and a VL comprising SEQ ID NO: 8. In some embodiments, the antibody that binds human IL-13 comprises a heavy chain comprising SEQ ID NO: 9, and a light chain comprising SEQ ID NO: 10. In some embodiments, the antibody that binds human IL-13 is lebrikizumab.

In some embodiments, the antibody that binds human IL-13 is administered subcutaneously to the patient. In some embodiments, the antibody that binds human IL-13 is administered at 250 mg or 500 mg subcutaneously to the patient. In some embodiments, the antibody that binds human IL-13 is administered subcutaneously to the patient once every two weeks.

In some embodiments, the patient is treated with the antibody that binds human IL-13 for a treatment period of about 16-24 weeks. In some embodiments, the patient is treated with the antibody that binds human IL-13 for a treatment period of about 16 weeks. In some embodiments, the patient is treated with the antibody that binds human IL-13 for a treatment period of about 24 weeks. In some embodiments, the patient is treated with a loading dose of 500 mg of the antibody that binds human IL-13 at week 0 and week 2, and a subsequent dose of 250 mg of the antibody that binds human IL-13 once every two weeks for the rest of the treatment period.

In some embodiments, the patient is further treated for a maintenance period, e.g., up to 36 weeks. In some embodiments, the patient is treated with a maintenance dose of 250 mg of the antibody that binds human IL-13 once every two weeks during the maintenance period. In some embodiments, the patient is treated with a maintenance dose of 250 mg of the antibody that binds human IL-13 once every four weeks during the maintenance period. In some embodiments, the patient is treated with a maintenance dose of 250 mg of the antibody that binds human IL-13 once every eight weeks during the maintenance period.

In some embodiments, the methods provided herein further comprise determining one or more of the following characteristics of the patient before and after the treatment period and/or maintenance period: Eczema Area and Severity Index (EASI), Investigator Global Assessment (IGA), Face-Investigator's Global Assessment (F-IGA), Pruritus numeric rating scale (NRS) score, Modified Total Lesion Symptom Scale, Sleep Loss Scale, Skin Pain NRS score, Severity Scoring of Atopic Dermatitis (SCORAD), Dermatology Life Quality Index (DLQI) or Children Dermatology Life Quality Index (CDLQI) score, Atopic Dermatitis Control Tool (ADCT), Participant-Reported Satisfaction Question, Fitzpatrick Skin Phototype Assessment Scale, Work Productivity and Activity Impairment Questionnaire-Atopic Dermatitis (WPAI-AD), Patient-Oriented Eczema Measure (POEM), European Quality of Life-5 Dimensions-5 Levels (EQ-5D-5L), and PROMIS (Patient-Reported Outcomes Measurement Information System) Anxiety and Depression measures.

In some embodiments, the methods, uses, and pharmaceutical compositions described herein further comprise administrating one or more topical corticosteroids to the patient. Accordingly, also provided herein are antibodies that bind human IL-13 for administration in separate, simultaneous, or sequential combination with one or more topical corticosteroids. In some embodiments, the topical corticosteroid is triamcinolone acetonide, hydrocortisone, or a combination of triamcinolone acetonide and hydrocortisone. In some embodiments, the topical corticosteroids are administered concomitantly with the antibody.

### BRIEF DESCRIPTION OF THE DRAWINGS

FIG. 1 is a schematic diagram of the study design described in Example 1.

FIG. 2 is a schematic diagram of the study design described in Example 2. n=number; Q2W=once every 2 weeks; SC=subcutaneous; W=week. The Screening window is 30 days. Lebrikizumab Loading Dose of 500 mg is administered SC at baseline (Week 0) and Week 2. The safety follow-up visit occurs at Week 34 or approximately 12 weeks after last study intervention injection.

### DETAILED DESCRIPTION

Provided herein are methods, uses and pharmaceutical compositions of anti-IL-13 antibodies for treating atopic dermatitis. In some embodiments, provided herein are methods and uses of anti-IL-13 antibodies for treating atopic dermatitis, e.g., in a patient who has been previously administered dupilumab.

In one aspect, provided herein are methods of treating moderate to severe atopic dermatitis in a patient in need thereof who has been previously administered dupilumab, which comprise administering to the patient an antibody that binds human IL-13. In some embodiments, provided herein are methods for treating moderate to severe atopic dermatitis, which comprise selecting a patient who has been previously administered dupilumab, and administering to the patient an antibody that binds human IL-13. In some embodiments, the patients stopped dupilumab due to no response, partial response, or loss of response. In some embodiments, the patients stopped dupilumab due to intolerance or adverse events. In some embodiments, the patients stopped dupilumab for other reasons. In some embodiments, the patient stopped dupilumab for at least four weeks. In some embodiments, the patient stopped dupilumab for at least eight weeks.

In some embodiments, provided herein are methods for treating moderate to severe atopic dermatitis, which comprise selecting a patient who has moderate to severe atopic dermatitis and has been previously administered dupilumab and stopped dupilumab due to non-response, partial response, or loss of response, and administering to the patient an antibody that binds human IL-13. In some embodiments, the patient stopped dupilumab for at least four weeks. In some embodiments, the patient stopped dupilumab for at least eight weeks.

In some embodiments, provided herein are methods for treating moderate to severe atopic dermatitis, which comprise selecting a patient who has moderate to severe atopic dermatitis and has been previously administered dupilumab and stopped dupilumab due to intolerance or adverse event, and administering to the patient an antibody that binds human IL-13. In some embodiments, the patient stopped dupilumab for at least four weeks. In some embodiments, the patient stopped dupilumab for at least eight weeks.

In some embodiments, the patient has moderate to severe atopic dermatitis for at least a year. In some embodiments, the moderate to severe atopic dermatitis is determined by the American Academy of Dermatology Consensus Criteria. In some embodiments, the patient has an Eczema Area and Severity Index (EASI) score of 16 or greater, an Investigator Global Assessment (IGA) score of 3 or greater, and more than 10% of body surface area (BSA) affected by atopic dermatitis, before administration of the antibody. In some embodiments, the patient had inadequate response to topical corticosteroids. In some embodiments, the patient is 12 years of age or older.

Also provided herein are methods for treating moderate to severe atopic dermatitis, which comprise selecting a patient who (i) is 12 years of age or older; (ii) has chronic atopic dermatitis according to American Academy of Dermatology Consensus Criteria for more than a year; (iii) has an EASI score of 16 or greater; (iv) has an IGA score of 3 or greater; (v) has more than 10% of body surface area affected by atopic dermatitis; (vi) had inadequate response to topical corticosteroids; and (vii) has been previously administered dupilumab, wherein the patient stopped dupilumab due to (1) non-response, partial response, or loss of response, or (2) intolerance or adverse event; and administering to the patient an antibody that binds human IL-13. In some embodiments, the patient stopped dupilumab due to non-response, partial response, or loss of response. In some embodiments, the patient stopped dupilumab due to intolerance or adverse event. In some embodiments, the patient stopped dupilumab due to non-response or partial response. In some embodiments, the patient stopped dupilumab due to loss of response. In some embodiments, the patient stopped dupilumab for at least four weeks. In some embodiments, the patient stopped dupilumab for at least eight weeks.

In some embodiments, the moderate to severe atopic dermatitis can be determined by the American Academy of Dermatology Criteria for the Diagnosis and Assessment of Atopic Dermatitis (Eichenfield et al., JAm Acad Dermatol. 2014; 70(2):338-351). The essential features that must be present for the diagnosis of AD include pruritus and acute, subacute, or chronic eczema consisting of typical morphology and age-specific patterns, or chronic or relapsing history. Age-specific patterns include facial, neck, and extensor involvement in infants and children; current or prior flexural lesions in any age group; and sparing of the groin and axillary regions. The important features are seen in most cases and support the diagnosis of AD include early age of onset, atopy (personal and/or family history, and/or IgE reactivity), and xerosis. The clinical associations that help to suggest the diagnosis of AD but are too nonspecific to be used for defining or detecting AD for research and epidemiologic studies include atypical vascular responses, such as, facial pallor, white dermographism, and delayed blanch response, keratosis pilaris or pityriasis alba or hyperlinear palms or ichthyosis, ocular or periorbital changes, other regional findings such as, perioral changes or periauricular lesions, and perifollicular accentuation or lichenification or prurigo lesions. A diagnosis of AD also depends on excluding conditions, such as scabies, seborrheic dermatitis, contact dermatitis (irritant or allergic), ichthyoses, cutaneous T-cell lymphoma, psoriasis, photosensitivity dermatoses, immune deficiency diseases, or erythroderma of other causes.

The severity of atopic dermatitis can also be determined by the “Hanifin and Rajka criteria” as described in Hanifin and Rajka diagnostic criteria are described in Acta Derm Venereol (Stockh) 1980; Suppl 92:44-7; or the “Rajka and Langeland criteria,” as described in Rajka G and Langeland T, Acta Derm Venereol (Stockh) 1989; 144(Suppl):13-4.

The antibodies suitable for use in the methods and uses provided herein have been described previously, e.g., WO2005062967. In some embodiments, the antibody binds human IL-13 with high affinity and blocks signaling through the active IL-4Ralpha/IL-13Ralpha1 heterodimer. In some embodiments, the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6. In some embodiments, the antibody that binds human IL-13 comprises a VH comprising SEQ ID NO: 7, and a VL comprising SEQ ID NO: 8. In some embodiments, the antibody that binds human IL-13 comprises a heavy chain comprising SEQ ID NO: 9, and a light chain comprising SEQ ID NO: 10. In some embodiments, the antibody that binds human IL-13 is lebrikizumab. The amino acid sequences of lebrikizumab are provided in Table 1. C-terminal clipping of IgG antibodies could occur where one or two C-terminal amino acids are removed from the heavy chain of the IgG antibodies. For example, if a C-terminal lysine (K) is present, it may be truncated or clipped off from the heavy chain. A penultimate glycine (G) may also be truncated or clipped off from the heavy chain as well. Modification of N-terminal amino acid of IgG could also occur. For example, the N-terminal glutamine (Q) or glutamic acid (E) can cyclize into pyro-glutamate (pE) spontaneously. SEQ ID NO: 9 reflects these potential modifications of lebrikizumab heavy chain.

### Table 1. Lebrikizumab Sequences

TABLE 1
Lebrikizumab Sequences

| SEQ | | |
|---|---|---|
| ID NO: | Description | Sequence |
| 1 | Lebrikizumab | AYSVN |
| | HCDR1 | |
| 2 | Lebrikizumab | MIWGDGKIVYNSALKS |
| | HCDR2 | |
| 3 | Lebrikizumab | DGYYPYAMDN |
| | HCDR3 | |
| 4 | Lebrikizumab | RASKSVDSYGNSFMH |
| | LCDR1 | |
| 5 | lebrikizumab | LASNLES |
| | LCDR2 | |
| 6 | Lebrikizumab | QQNNEDPRT |
| | LCDR3 | |
| 7 | Lebrikizumab | VTLRESGPALVKPTQTLTLTCTVSGFSLSAYSVNWIR |
| | heavy chain | QPPGKALEWLAMIWGDGKIVYNSALKSRLTISKDTSK |
| | variable region | NQVVLTMTNMDPVDTATYYCAGDGYYPYAMDNWG |
| | (VH) | QGSLVTVSS |
| 8 | Lebrikizumab | DIVMTQSPDSLSVSLGERATINCRASKSVDSYGNSF |
| | light chain | MHWYQQKPGQPPKLLIYLASNLESGVPDRFSGSGS |
| | variable region | GTDFTLTISSLQAEDVAVYYCQQNNEDPRTFGGGTK |
| | (VL) | VEIK |
| 9 | Lebrikizumab | Xaa1VTLRESGPALVKPTQTLTLTCTVSGFSLSAYSV |
| | heavy chain | NWIRQPPGKALEWLAMIWGDGKIVYNSALKSRLTISK |
| | (HC) | DTSKNQVVLTMTNMDPVDTATYYCAGDGYYPYAMD |
| | | NWGQGSLVTVSSASTKGPSVFPLAPCSRSTSESTAA |
| | | LGCLVKDYFPEPVTVSWNSGALTSGVHTFPAVLQSS |
| | | GLYSLSSVVTVPSSSLGTKTYTCNVDHKPSNTKVDK |
| | | RVESKYGPPCPPCPAPEFLGGPSVFLFPPKPKDTLMI |
| | | SRTPEVTCVVVDVSQEDPEVQFNWYVDGVEVHNAK |
| | | TKPREEQFNSTYRVVSVLTVLHQDWLNGKEYKCKVS |
| | | NKGLPSSIEKTISKAKGQPREPQVYTLPPSQEEMTKN |
| | | QVSLTCLVKGFYPSDIAVEWESNGQPENNYKTTPPV |
| | | LDSDGSFFLYSRLTVDKSRWQEGNVFSCSVMHEAL |
| | | HNHYTQKSLSLSLXaa2Xaa3 |
| | | wherein Xaa1 is Q, pE, or absent; |
| | | Xaa2 is G or absent; |
| | | Xaa3 is K or absent. |
| 10 | Lebrikizumab | DIVMTQSPDSLSVSLGERATINCRASKSVDSYGNSF |
| | light chain | MHWYQQKPGQPPKLLIYLASNLESGVPDRFSGSGS |
| | (LC) | GTDFTLTISSLQAEDVAVYYCQQNNEDPRTFGGGTK |
| | | VEIKRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNRG |
| | | EC |

The anti-IL-13 antibodies, e.g., lebrikizumab, can be formulated with suitable carriers or excipients into a pharmaceutical composition that is suitable for administration to patients. For example, the anti-IL-13 antibodies, e.g., lebrikizumab, can be formulated in a pharmaceutical composition as described in WO 2013/066866. The pharmaceutical composition can comprise 100 mg, 150 mg, 200 mg, 250 mg, 300 mg, 350 mg, 400 mg, 450 mg, or 500 mg of the anti-IL-13 antibody. In some embodiments, the pharmaceutical composition comprises 250 mg or 500 mg of the anti-IL-13 antibody. In some embodiments, the anti-IL-13 antibody concentration in the pharmaceutical composition is between 100 mg/mL and 150 mg/mL, e.g., 125 mg/mL. The pharmaceutical composition can also comprise 5 mM-40 mM histidine acetate buffer, pH 5.4 to 6.0. In some embodiments, the pharmaceutical composition further comprises a polyol (e.g., sugar) that has a concentration between 100 mM and 200 mM, and/or a surfactant (e.g., polysorbate 20) that has a concentration of 0.01%-0.1%. In one embodiment, the pharmaceutical composition comprises 125 mg/mL of anti-IL-13 antibody (e.g., lebrikizumab), 20 mM histidine acetate buffer, pH 5.7, 175 mM sucrose and 0.03% polysorbate 20.

In some embodiments, the anti-IL-13 antibody is administered subcutaneously to the patient. The anti-IL-13 antibody can be administered to the patient at a dosing frequency of about once a week, once every two weeks, once every three weeks, once every four weeks, once every five weeks, once every six weeks, or once every eight weeks. In some embodiments, the anti-IL-13 antibody is administered to the patient once every two weeks. In some embodiments, the anti-IL-13 antibody is administered to the patient once every four weeks. In some embodiments, the anti-IL-13 antibody is administered to the patient once every eight weeks.

In some embodiments, the anti-IL-13 antibody is administered to the patient using a subcutaneous administration device. The subcutaneous administration device can be selected from a prefilled syringe, disposable pen injection device, microneedle device, microinfuser device, needle-free injection device, or autoinjector device. Various subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). In some embodiments, the subcutaneous administration device is an autoinjector device described in WO 2008/112472, WO 2011/109205, WO 2014/062488, and/or WO 2016/089864.

In some embodiments, the patient is treated with the anti-IL-13 antibody for a treatment period of about 16-24 weeks, e.g., 16 weeks, 18 weeks, 20 weeks, 22 weeks, or 24 weeks. In some embodiments, the patient is treated with the anti-IL-13 antibody for a treatment period of about 16 weeks. In some embodiments, the patient is treated with the anti-IL-13 antibody for a treatment period of about 24 weeks. In some embodiments, the patient is treated with a loading dose of 500 mg of the anti-IL-13 antibody at week 0 and week 2, and a subsequent dose of 250 mg of the anti-IL-13 antibody once every two weeks for the rest of the treatment period.

In some embodiments, the patient is further treated with the anti-IL-13 antibody for a maintenance period, e.g., up to 36 weeks (e.g., about 4 weeks, 8 weeks, 12 weeks, 16 weeks, 20 weeks, 24 weeks, 28 weeks, 32 weeks, 36 weeks). In some embodiments, the patient is treated with a maintenance dose of 250 mg of the anti-IL-13 antibody once every two weeks during the maintenance period. In some embodiments, the patient is treated with a maintenance dose of 250 mg of the anti-IL-13 antibody once every four weeks during the maintenance period. In some embodiments, the patient is treated with a maintenance dose of 250 mg of the anti-IL-13 antibody once every eight weeks during the maintenance period.

Before, during, and after the treatment period and/or maintenance period, the patient can be assessed for one or more characteristics of the Atopic Dermatitis Disease Severity Measures (ADDSM), which determine certain signs, symptoms, features, or parameters that have been associated with atopic dermatitis and that can be quantitatively or qualitatively assessed. Exemplary ADDSM include, but are not limited to, Eczema Area and Severity Index (EASI), Investigator Global Assessment (IGA), Face-Investigator's Global Assessment (F-IGA), Pruritus numeric rating scale (NRS) score, Modified Total Lesion Symptom Scale, Sleep Loss Scale, Skin Pain NRS score, Severity Scoring of Atopic Dermatitis (SCORAD), Dermatology Life Quality Index (DLQI) or Children Dermatology Life Quality Index (CDLQI) score, Atopic Dermatitis Control Tool (ADCT), Participant-Reported Satisfaction Question, Fitzpatrick Skin Phototype Assessment Scale, Work Productivity and Activity Impairment Questionnaire-Atopic Dermatitis (WPAI-AD), Patient-Oriented Eczema Measure (POEM), European Quality of Life-5 Dimensions-5 Levels (EQ-5D-5L), and PROMIS (Patient-Reported Outcomes Measurement Information System) Anxiety and Depression measures.

The ADDSM can be measured at baseline (prior to the administration of the antibody) and at one or more time points after administration of the antibody. For example, an ADDSM may be measured at the end of week 1, week 2, week 3, week 4, week 5, week 6, week 7, week 8, week 9, week 10, week 11, week 12, week 13, week 14, week 15, week 16, or longer after the initial treatment with the antibody. The difference between the value of the ADDSM at a particular time point following initiation of treatment and the value of the ADDSM at baseline is used to establish whether there has been an improvement (e.g., a reduction) in the ADDSM.

The “Eczema Area and Severity Index” or “EASI” is an investigator-reported, 20-item scale that evaluates 2 dimensions of AD: extent of disease at 4 body regions (head or neck, trunk, upper and lower extremities) and 4 clinical signs (erythema, induration or papulation, excoriation, and lichenification). The clinical signs are assessed for severity on a scale of 0 (absent) to 3 (severe). The scores are added up for each of the 4 body regions. The assigned percentages of BSA for each section of the body are 10% for head or neck, 20% for upper extremities, 30% for trunk, and 40% for lower extremities, respectively. Each subtotal score is multiplied by the BSA represented by that region. In addition, an area score of 0 to 6 is assigned for each body region, depending on the percentage of AD-affected skin in that area: 0 (none), 1 (1% to 9%), 2 (10% to 29%), 3 (30% to 49%), 4 (50% to 69%), 5 (70% to 89%), or 6 (90% to 100%). Each of the body area scores is multiplied by the area affected. The resulting EASI ranges from 0 to 72 points, with the highest score indicating worse severity of AD (Hanifin et al., Exp Dermatol. 2001; 10:11-18).

The “Investigator Global Assessment” or “IGA” is an investigator-reported, single-item scale that rates the severity of the participant's AD (Simpson E, et al. J Am Acad Dermatol. 2020; 83(3):839-846). The IGA is composed of a 5-point scale ranging from 0 (clear) to 4 (severe), and a score is selected using descriptors that best describe the overall appearance of the lesions at a given time point (see Table 2).

TABLE 2
Investigator Global Assessment

| Score | Grade | Definition |
|---|---|---|
| 0 | Clear | Minor, residual discoloration; no erythema or |
| | | induration/papulation; no oozing/crusting; |
| | | no edema. |
| 1 | Almost | Trace, faint pink erythema with barely perceptible |
| | clear | induration/papulation and no oozing/crusting; |
| | | no edema. |
| 2 | Mild | Faint-pink erythema with papulation and edema |
| | | perceptible upon palpation and no oozing/crusting; |
| | | minimal induration. |
| 3 | Moderate | Pink-red erythema with definite edema of skin |
| | | papules and plaques; there may be some oozing/ |
| | | crusting; palpable induration. |
| 4 | Severe | Deep/bright red erythema with significant swelling |
| | | and obvious raised borders of papules and plaques |
| | | with oozing/crusting; significant induration. |

The body surface area (BSA) is an investigator-reported assessment, which estimates the extent of disease or skin involvement of a participant's AD. BSA is expressed as a percentage of total body surface and reported by body location. BSA is determined by an investigator using the patient palm is about 1% BSA rule. The recall period of this assessment is present time.

The Face-Investigator's Global Assessment (F-IGA) is an investigator-administered, single-item scale that rates the severity of the participant's AD on the face. The F-IGA is composed of a 5-point scale ranging from 0 (clear) to 4 (severe), and a score is selected using descriptors that best describe the overall appearance of the facial lesions at a given time point.

The “Severity Scoring of Atopic Dermatitis” or “SCORAD” index is an investigator- and participant-reported, 9-item assessment in both adults and adolescent participants that assesses 3 aspects: extent, severity, and subjective symptoms (Stalder J., Taieb A., Consensus report of the European Task Force on Atopic Dermatitis. Dermatology. 1993; 186(1):23-31). The extent (1-item) of AD is assessed by the investigator as a percentage of each defined body area and reported as the sum of all areas. The maximum score is 100%. The recall period of this scale is present time. The severity of 6 specific symptoms of AD is assessed by the investigator: erythema, edema or papulation, oozing or crusts, excoriation, lichenification, and dryness are assessed by the investigator using a 4-point scale (that is, none=0, mild=1, moderate=2, severe=3) with a maximum possible total of 18 points. The recall period of this scale is present time. The subjective symptoms of pruritus and sleep loss (2 items) are assessed by the participant via a 10-cm visual analog scale. The symptoms (itch and sleeplessness) are recorded by the participant or caregiver on a visual analogue scale, where 0 is no symptoms and 10 is the worst imaginable symptom, with a maximum possible score of 20. The recall period of this scale is over the last 3 days. The maximum possible SCORAD score calculated based on the above 3 aspects is 103. The higher scores indicate poorer or more severe condition.

The Modified Total Lesion Symptom Scale is an investigator-reported scale that combines the evaluation of hand eczema (HE) lesion severity (erythema, edema, desquamation, fissures, hyperkeratosis or lichenification, and vesicles) with the intensity of pruritus or pain (Bissonnette et al., J Eur Acad Dermatol Venereol. 2010; 24(s3):1-20) to assess the severity of symptoms. This composite score assigns 0 (mild) to 3 (severe) to each component, giving a maximum disease severity of 21. The recall period for this scale is the present time. It has been used as a secondary endpoint in studies investigating alitretinoin in HE (Ruzicka et al., Br J Dermatol. 2008; 158(4):808-817; Fowler et al., J Drugs Dermatol. 2014; 13(10):1198-204).

Pruritus Numerical Rating Scale (NRS) is a participant-reported, single-item, daily, 11-point scale. The Pruritus NRS is used by participants to rate their worst itch severity over the past 24 hours with 0 indicating “No itch” and 10 indicating “Worst itch imaginable.” Assessments will be recorded daily by the participant. The minimal clinically important change is 3 points. (Yosipovitch et al., Br J Dermatol. 2019; 181(4):761-769). Participants will record the pruritus assessments daily using an electronic diary at home. The initial electronic diary entries for Pruritus NRS should be completed a minimum of 4 of 7 days before baseline.

The Skin Pain NRS is a participant-reported, 11-point horizontal scale anchored at 0 and 10, with 0 representing “no pain” and 10 representing “worst pain imaginable.” Overall severity of a participant's skin pain is indicated by selecting the number that best describes the worst level of skin pain in the past 24 hours (Newton L, et al. J Patient Rep Outcomes. 2019 Jul. 16; 3:42; Silverberg et al., Health Qual Life Outcomes. 2021; 19(1):247). Assessment will be recorded daily by the participant.

The Sleep-Loss Scale is a participant-reported, single-item, daily scale that measures the extent of sleep loss due to interference of itch over the last night. The Sleep-Loss Scale is rated based on a 5-point Likert scale (0 [not at all] to 4 [unable to sleep at all]). Assessment is recorded daily by the participant. The minimal clinically important change is 3 points (Yosipovitch et al. 2021, Content Validity And Assessment Of The Psychometric Properties And Score Interpretation Of A Pruritus Numeric Rating Scale In Atopic Dermatitis. Revolutionizing Atopic Dermatitis—December 2021). Participants record the pruritus assessments daily using an electronic diary at home. The initial electronic diary entries for Sleep-Loss Scale are completed a minimum of 4 of 7 days before baseline.

The Dermatology Life Quality Index (DLQI) is a participant-reported, 10-item, QoL questionnaire in adults that covers 6 domains (symptoms and feelings, daily activities, leisure, work and school, personal relationships, and treatment). The recall period of this scale is over the “last week.” Response categories include “not at all,” “a little”, “a lot,” and “very much,” with corresponding scores of 0, 1, 2, and 3, respectively, and unanswered (or “not relevant”) responses scored as 0. Scores range from 0 to 30 with higher scores indicating greater impairment of QoL. A DLQI total score of 0 to 1 is considered as having no effect on a participant's HRQoL (Hongbo et al. 2005), and a 4-point change from baseline is considered as the minimal clinically important difference threshold (Khilji et al. 2002, Br J Dermatol.; 147:25-54; Basra et al. 2015, Dermatology.; 230(1):27-33).

For adolescents below the age of 16 at baseline, the Children DLQI (CDLQI) is employed which is based on a set of 10 questions different from those of the DLQI (Lewis-Jones MS, Finlay A Y. British Journal of Dermatology, 1995; 132:942-949), and should continue to complete the CDLQI for the duration of the study.

The Atopic Dermatitis Control Tool (ADCT) is a patient-reported, simple, brief tool for adults and adolescents that evaluates 6 symptoms and effects associated with AD over the past week: overall severity of symptoms, days with intense episodes of itching, intensity of bother, problem with sleep, impact on daily activities, and impact on mood or emotions. Each of the 6 Atopic Dermatitis Control Tool items has a score range from 0 (no problem) to 4 (worst), rating the severity of each concept; the total score ranges from 0 to 24, which is the summation of the responses to all the items. A score of ≥7 points was derived as the threshold to identify participants “not in control.” The threshold for meaningful within-person change was estimated to be 5 points (Simpson et al., BMC Dermatol. 2019; 19(1):15; Pariser et al., Curr Med Res Opin. 2020; 36(3):367-376).

The Participant-Reported Satisfaction Question asks “How satisfied are you with this treatment's ability to treat your skin condition?” The response options range from 1 (not satisfied) to 5 (completely satisfied).

The Patient-Oriented Eczema Measure (POEM) is a 7-item scale that assesses disease severity in children and adults. Participants respond to questions about the frequency of 7 symptoms (itching, sleep disturbance, bleeding, weeping/oozing, cracking, flaking, and dryness/roughness) over the past week. Response categories include “No days,” “1 to 2 days,” “3 to 4 days,” “5 to 6 days,” and “Every day” with corresponding scores of 0, 1, 2, 3, and 4, respectively. Scores range from 0 to 28, with higher total scores indicating greater disease severity (Charman et al., Arch Dermatol. 2004; 140(12):1513-1519). A high score is indicative of a poor quality of life.

The European Quality of Life-5 Dimensions-5 Levels (EQ-5D-5L, EuroQol Research Foundation) is a standardized 5-item self-administered instrument for use as a measure of health outcome. It provides a simple descriptive profile and a single index value for health status that can be used in the clinical and economic evaluation of health care as well as population health surveys. The European Quality of Life-5 Dimensions-5 Levels assesses 5 dimensions of health: mobility, self-care, usual activities, pain/discomfort, and anxiety/depression. The 5L version, scores each dimension at 5 levels: no problems, slight problems, moderate problems, severe problems, and unable to perform/extreme problems. A total of 3125 health states is possible. In addition to the health profile, a single health state index value can be derived based on a formula that attaches weights to each of the levels in each dimension. This index value ranges between less than 0 (where 0 is a health state equivalent to death; negative values are valued as worse than dead) to 1 (perfect health). In addition, the EQ visual analog scale records the respondent's self-rated health status on a vertical graduated (0 to 100) visual analog scale. The participant rates his/her perceived health from 0 (the worst imaginable health) to 100 (the best imaginable health). In conjunction with the health state data, it provides a composite picture of the respondent's health status.

The Fitzpatrick Skin Phototype Assessment Scale is a clinician-rated scale and is based on a participant's cutaneous reaction to sun exposure and baseline skin pigmentation. Fitzpatrick skin phototypes range from I to VI, with a score of I indicating “always burns, does not tan, white skin tone” and a score of VI indicating “never burns, tans very easily, skin color black” (High et al. 2012, Dermatology (3rd ed.). China: Elsevier; 2012: pp. 8). Investigators will follow the descriptive terms included in this protocol when recording the Fitzpatrick skin phototype. The sun sensitivity measure will represent the Fitzpatrick Scale score for each participant. The recall period for this scale is the present time.

The Work Productivity and Activity Impairment Questionnaire-Atopic Dermatitis (WPAI-AD) is a participant-reported, 6-item questionnaire that records impairment due to AD over the past 7 days in adolescents and adults. The Work Productivity and Activity Impairment Questionnaire-Atopic Dermatitis consists of 6 items grouped into 4 domains: absenteeism (work time missed), presenteeism (impairment at work or reduced on the job effectiveness), work productivity loss (overall work impairment or absenteeism plus presenteeism), and activity impairment. Absenteeism, presentism, and work productivity will only be reported by those who are currently employed (working for pay) at the time of the scale completion. The recall period for the scale is over the past 7 days. Scores are calculated as impairment percentages (Reilly et al., Pharmacoeconomics. 1993; 4(5):353-365), with higher scores indicating greater impairment and less productivity.

PROMIS (Patient-Reported Outcomes Measurement Information System) is a set of person-centered measures that evaluates and monitors physical, mental, and social health in adults and children (PROMIS WWW). The PROMIS measures include Anxiety and Depression short forms, which assess the participants' symptoms over the previous week. It can be used with the general population and with individuals living with chronic conditions.

The PROMIS Anxiety measures assesses the following items: self-reported fear (fearfulness, panic), anxious misery (worry, dread), hyperarousal (tension, nervousness, restlessness), and somatic symptoms related to arousal (racing heart, dizziness) (PROMIS Anxiety 2019, available at https://www.healthmeasures.net/images/PROMIS/manuals/PROMIS_Anxiety_Scoring_Manual. pdf). The PROMIS Anxiety Short Form (8 questions, 8a v2.0) is available in a pediatric self-report (ages 8 to <18 years) and for parents/caregivers serving as proxy reporters for their children (youth ages≥5 years). Children aged<5 years do not complete this assessment. Both pediatric self-report and proxy-report versions assess anxiety “in the past 7 days.” Response options range from 1=Never; 2=Rarely; 3=Sometimes; 4=Often; to 5=Almost always. Total raw scores are converted to T-Scores with higher scores representing greater anxiety.

The PROMIS Depression measures assesses the following items: self-reported negative mood (sadness, guilt), views of self (self-criticism, worthlessness), social cognition (loneliness, interpersonal alienation), and decreased positive affect and engagement (loss of interest, meaning, and purpose) (PROMIS Depression 2019, available at https://www.healthmeasures.net/images/PROMIS/manuals/PROMIS_Depression_Scoring_Man ual.pdf). The PROMIS Depression Short Form (8a v2.0 and 6a v2.0) is available in a pediatric self-report (ages 8 to <18 years) and for parents/caregivers serving as proxy reporters for their children (youth ages≥5 years). Children aged<5 years do not complete this assessment. Both pediatric self-report and proxy-report versions assess depression “in the past 7 days.” Response options range from 1=Never; 2=Rarely; 3=Sometimes; 4=Often; to 5=Almost always. Total raw scores are converted to T-Scores with higher scores representing greater depression.

In some embodiments, the EASI score of the patient is determined after the treatment period, e.g., at Week 16 or Week 24. In some embodiments, the patient's EASI score determined after the treatment period is reduced by 50% or greater compared to the EASI score determined prior to administration of the first loading dose of the antibody, which means the patient has achieved “EASI 50”. In some embodiments, the patient's EASI score determined after the treatment period is reduced by 75% or greater compared to the EASI score determined prior to administration of the first loading dose of the antibody, which means the patient has achieved “EASI 75”. In some embodiments, the patient's EASI score determined after the treatment period is reduced by 90% or greater compared to the EASI score determined prior to administration of the first loading dose of the antibody, which means the patient has achieved “EASI 90”.

In some embodiments, the IGA score of the patient is determined after the treatment period. In some embodiments, the patient's IGA score determined after the treatment period is 0 or 1 and the IGA score determined after the treatment period is reduced by 2 points or greater compared to the IGA score determined prior to administration of the first loading dose of the antibody.

In some embodiments, the pruritus NRS score of the patient is determined after the treatment period. In some embodiments, the patient's pruritus NRS score determined after the treatment period is reduced by 4 points or greater compared to the pruritus NRS score determined prior to administration of the first loading dose of the antibody.

In another aspect, provided herein is an antibody that binds human IL-13 for use in the treatment of moderate to severe atopic dermatitis in a patient who has been previously administered dupilumab. In some embodiments, provided herein is an antibody that binds human IL-13 for use in the treatment of moderate to severe atopic dermatitis in a patient who has been previously administered dupilumab and stopped dupilumab due to non-response, partial response, or loss of response. In some embodiments, provided herein is an antibody that binds human IL-13 for use in the treatment of moderate to severe atopic dermatitis in a patient who has been previously administered dupilumab and stopped dupilumab due to intolerance or adverse event. In some embodiments, the patient: (1) is 12 years of age or older; (2) has chronic atopic dermatitis according to American Academy of Dermatology Consensus Criteria for more than a year; (3) has an EASI score of 16 or greater; (4) has an IGA score of 3 or greater; (5) has more than 10% of BSA affected by atopic dermatitis; and (6) had inadequate response to topical corticosteroids. In some embodiments, the antibody is for subcutaneous administration to the patient.

In another aspect, provided herein are uses of an antibody that binds human IL-13 in the manufacture of a medicament for the treatment of moderate to severe atopic dermatitis in a patient who has been previously administered dupilumab. Also provided herein are uses of an antibody that binds human IL-13 in the manufacture of a medicament for the treatment of moderate to severe atopic dermatitis in a patient who has been previously administered dupilumab and stopped dupilumab due to non-response, partial response, or loss of response. Also provided herein are uses of an antibody that binds human IL-13 in the manufacture of a medicament for the treatment of moderate to severe atopic dermatitis in a patient who has been previously administered dupilumab and stopped dupilumab due to intolerance or adverse event. In some embodiments, the patient: (1) is 12 years of age or older; (2) has chronic atopic dermatitis according to American Academy of Dermatology Consensus Criteria for more than a year; (3) has an EASI score of 16 or greater; (4) has an IGA score of 3 or greater; (5) has more than 10% of BSA affected by atopic dermatitis; and (6) had inadequate response to topical corticosteroids. In some embodiments, the antibody is for subcutaneous administration to the patient.

In some embodiments, the methods and uses described herein further comprise administrating one or more topical corticosteroids to the patient. Exemplary topical corticosteroids include, but are not limited to, triamcinolone acetonide, hydrocortisone, and a combination of triamcinolone acetonide and hydrocortisone. Triamcinolone acetonide is typically formulated at a concentration of 0.1% in a cream, and hydrocortisone is typically formulated at a concentration of 1% or 2.5% in a cream. Certain topical corticosteroids are considered very high potency such as, for example, betamethasone dipropionate, clobetasol propionate, diflorasone diacetate, fluocinonide, and halobetasol propionate. Certain topical corticosteroids are considered high potency such as, for example, amcinonide, desoximetasone, halcinonide, and triamcinolone acetonide. Certain topical corticosteroids are considered medium potency, such as, for example, betamethasone valerate, clocortolone pivalate, fluocinolone acetonide, flurandrenolide, fluocinonide, fluticasone propionate, hydrocortisone butyrate, hydrocortisone valerate, mometasone furoate, and prednicarbate. Certain topical corticosteroids are considered low potency, such as, for example, alclometasone dipropionate, desonide, and hydrocortisone. TCS may be applied to affected areas once daily, twice daily, three times per day, or as needed. In some embodiments, the patient is inadequately controlled on topical corticosteroids. In some embodiments, the topical corticosteroid is triamcinolone acetonide, hydrocortisone, or a combination of triamcinolone acetonide and hydrocortisone. In some embodiments, the topical corticosteroids are administered concomitantly or sequentially with the anti-IL-13 antibody. In some embodiments, the topical corticosteroids are administered concomitantly with the anti-IL-13 antibody. Accordingly, also provided herein are antibodies that bind human IL-13 for administration in separate, simultaneous, or sequential combination with one or more topical corticosteroids.

As used herein, the term “a,” “an,” “the” and similar terms used in the context of the present disclosure (especially in the context of the claims) are to be construed to cover both the singular and plural unless otherwise indicated herein or clearly contradicted by the context.

The term “about” as used herein, means in reasonable vicinity of the stated numerical value, such as plus or minus 10% of the stated numerical value.

The term “administer” or “administering” as used herein, refers to directly administer an antibody, compound, or composition to a subject, including treat a subject with an antibody, compound, or composition.

The term “adverse event” as used herein, refers to any untoward medical occurrence in a participant administered a pharmaceutical product and which does not necessarily have a causal relationship with the study intervention. An adverse event can therefore be any unfavorable and unintended sign (including an abnormal laboratory finding), symptom, or disease (new or exacerbated) temporally associated with the use of a medicinal product, whether or not related to the medicinal product.

The term “antibody,” as used herein, refers to an immunoglobulin molecule that binds an antigen. Embodiments of an antibody include a monoclonal antibody, polyclonal antibody, human antibody, humanized antibody, chimeric antibody, or conjugated antibody. The antibodies can be of any class (e.g., IgG, IgE, IgM, IgD, IgA) and any subclass (e.g., IgG1, IgG2, IgG3, IgG4).

An exemplary antibody is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); AI-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Exemplary embodiments of antibodies of the present disclosure also include antibody fragments or antigen-binding fragments, which comprise at least a portion of an antibody retaining the ability to specifically interact with an antigen such as Fab, Fab′, F(ab′)₂, Fv fragments, scFv, scFab, disulfide-linked Fvs (sdFv), a Fd fragment and linear antibodies.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

The term “flare” as used herein refers to increase in signs and/or symptoms leading to escalation of therapy, which can be an increase in dose, a switch to a higher-potency class of drug, or the start of another drug.

The term “high affinity” as used herein refers to the strength of binding of an antibody to human IL-13 with an equilibrium dissociation constant (K_{D}) of less than about 10⁻⁸ M, e.g., from 10⁻¹⁵ M to 10⁻⁸ M, or from 10⁻¹² M to 10⁻⁹ M.

The term “human IL-13” refers to human interleukin 13 (also known as P600), an immunoregulatory cytokine produced primarily by activated Th2 cells. There are two known human IL-13 isoforms: isoform a and isoform b. The term “human IL-13” as used herein refers collectively to all human IL-13 isoforms. The amino acid sequence for human IL-13 isoform a can be found at NCBI Accession No. NP_002179.2. The amino acid sequence for human IL-13 isoform b can be found at NCBI Accession No. NP_001341922.1.

The term “no response” or “non-response” as used herein refers to a patient's atopic dermatitis is not improved at all or only improved less than 25% to a treatment, for example, the patient's skin and/or itch is not improved at all or only improved less than 25% (e.g., measured by EASI score for skin and/or Pruritus NRS score for itch) at peak response to dupilumab.

The term “partial response” as used herein refers to a patient's atopic dermatitis is only improved partially to a treatment, for example, the patient's skin and/or itch is only improved between 25-50% (e.g., measured by EASI score for skin and/or Pruritus NRS score for itch) at peak response to dupilumab.

The term “loss of response” as used herein refers to a patient initially responded but lost response to a treatment, for example, the patient's skin and/or itch is initially improved (e.g., measured by EASI score for skin and/or Pruritus NRS score for itch) after dupilumab treatment, but then it worsened.

The term “intolerance” as used herein refers to unacceptable adverse events in response to a treatment. For example, a patient discontinued dupilumab due to anaphylaxis; new onset or worsening of ocular surface disease, including but not limited to conjunctivitis; new onset or worsening of facial dermatitis; new onset or worsening of injection site reaction; new onset or worsening of inflammatory arthritis; new onset or worsening of psoriasiform dermatitis; or new onset or worsening of alopecia areata.

The term “inadequate response” as used herein refers to inability to achieve good disease control of atopic dermatitis (e.g., not able to achieve IGA≤2 or EASI-75) after use of the treatment for the duration recommended by the product prescribing information, or flare of atopic dermatitis occurs while on the treatment.

The term “patient”, as used herein, refers to a human patient.

The term “topical corticosteroid” or “TCS”, as used herein includes Group I, Group II, Group Ill and Group IV topical corticosteroids. According to the Anatomical Therapeutic Chemical (ATC) Classification System of World Health Organization, the corticosteroids are classified as weak (Group 1), moderately potent (Group II) and potent (Group Ill) and very potent (Group IV), based on their activity as compared to hydrocortisone. Group IV TCS (very potent) are up to 600 times as potent as hydrocortisone and include clobetasol and halcinonide. Group Ill TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include, but are not limited to, clobetasone butyrate, and triamcinolone acetonide. Group I TCS (weak or mild) includes hydrocortisone, prednisolone, and methylprednisolone.

As used herein, “treatment”, “treat” or “treating” refers to all processes wherein there may be a slowing, controlling, delaying, or stopping of the progression of the disorders or disease disclosed herein, or ameliorating disorder or disease symptoms, but does not necessarily indicate a total elimination of all disorder or disease symptoms. Treatment includes administration of a protein or nucleic acid or vector or composition for treatment of a disease or condition in a patient, particularly in a human.

### EXAMPLES

### Example 1. A Randomized, Double-Blind, Placebo-Controlled Trial to Evaluate the Efficacy and Safety of Lebrikizumab when Used in Combination with Topical Corticosteroid (TCS) Treatment in Patients with Moderate-to-Severe Atopic Dermatitis (KGAD)

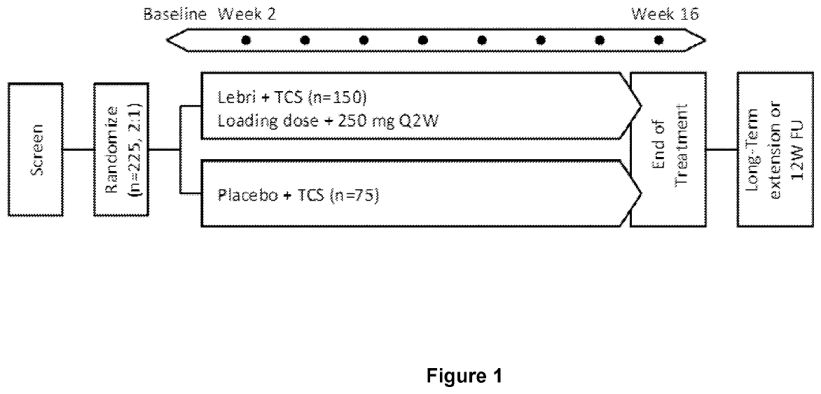

This is a randomized, double-blind, placebo-controlled, parallel-group study which is 16 weeks in duration. The study is designed to evaluate the safety and efficacy of lebrikizumab when used in combination with TCS treatment compared with placebo in combination with TCS treatment for moderate-to-severe AD. The study design is shown in FIG. 1.

During the 16-week treatment period, approximately 225 patients were stratified and randomized 2:1 to treatment with either 250 mg lebrikizumab (loading dose of 500 mg given at Baseline (Week 0) and Week 2) or placebo by subcutaneous (SC) injection every 2 weeks (Q2W). All study drug injections were administered in the clinic. TCS treatment was initiated at Baseline in all patients and may be tapered or stopped, as needed, based on treatment response.

After completion of the Week 16 visit, patients are offered the option to continue treatment in a separate long-term extension (LTE) study. Patients who early terminate or choose not to enter the long-term extension study received a safety follow-up approximately 12 weeks after the last study drug injection.

### Study Objectives and Endpoints

### Primary Endpoints:

For FDA, the primary efficacy endpoint is the percentage of patients with an IGA score of 0 or 1 and a reduction ≥2-points from Baseline to Week 16.

For EMA, the co-primary endpoints are used as follows: (1) Percentage of patients with an IGA score of 0 or 1 and a reduction ≥2-points from Baseline to Week 16; (2) Percentage of patients achieving EASI-75 (≥75% reduction from Baseline in EASI score) at Week 16.

### Secondary Endpoints

Table 3 below lists the secondary endpoints of this study.

TABLE 3
Secondary Endpoints

| FDA | EMA |
|---|---|
| Percentage of patients achieving | Percentage of patients achieving EASI- |
| EASI-75 (≥75% reduction from | 90 (≥90% reduction from Baseline in |
| Baseline in EASI score) at Week 16 | EASI score) at Week 16 |
| Percentage of patients achieving | Percentage change in Pruritus Numerical |
| EASI-90 (≥90% reduction from | Rating Scale (NRS) score from Baseline |
| Baseline in EASI score) at Week 16 | to Week 16 |
| Percentage change in Pruritus | Percentage of patients with a Pruritus |
| Numerical Rating Scale (NRS) score | NRS of ≥4-points at Baseline who |
| from Baseline to Week 16 | achieve a ≥4-point reduction from |
| Percentage of patients with a Pruritus | Baseline to Week 16 |
| NRS of ≥4-points at Baseline who | Percentage of patients with a Pruritus |
| achieve a ≥4-point reduction from | NRS of ≥5-points at Baseline who |
| Baseline to Week 16 | achieve a ≥4-point reduction from |
| Percentage change in EASI score | Baseline to Week 16 |
| from Baseline to Week 16 | Percentage change in EASI score from |
| Change from Baseline to Week 16 in | Baseline to Week 16 |
| percent BSA | Percentage of patients achieving EASI- |
| Percentage of patients achieving | 90 at Week 4 |
| EASI-90 at Week 4 | Change from baseline in DLQI at Week 16 |
| Percentage change in Sleep-loss | Percentage of patients achieving ≥4- |
| score from Baseline to Week 16 | point improvement in DLQI from baseline |
| Change from Baseline in Sleep-loss | to Week 16 |
| score at Week 16 | Percentage change in Sleep-loss score |
| Percentage of patients with a Pruritus | from Baseline to Week 16 |
| NRS of ≥4-points at Baseline who | Change from Baseline in Sleep-loss |
| achieve a ≥4-point reduction from | score at Week 16 |
| Baseline to Week 4 | Proportion of TCS/TCI-free days from |
| Percentage of patients with a Pruritus | Baseline to Week 16 |
| NRS of ≥4-points at Baseline who | Time (days) to TCS/TCI-free use from |
| achieve a ≥4-point reduction from | Baseline to Week 16 |
| Baseline to Week 2 | Percentage of patients with a Pruritus |
| Percentage of patients with a Pruritus | NRS of ≥4-points at Baseline who |
| NRS of ≥4-points at Baseline who | achieve a ≥4-point reduction from |
| achieve a ≥4-point reduction from | Baseline to Weeks 1, 2 and 4 |
| Baseline to Week 1 | Percentage of patients with a Pruritus |
| Proportion of TCS/TCI-free days | NRS of ≥5-points at Baseline who |
| from Baseline to Week 16 | achieve a ≥4-point reduction from |
| Time (days) to TCS/TCI-free use | Baseline to Weeks 1, 2 and 4 |
| from Baseline to Week 16 | |

Statistical analyses are performed for the primary and secondary endpoints.

### Patient Population

Inclusion Criteria: Patients must meet all the following criteria to be eligible for this study:
1. Adults and adolescents (≥12 to <18 years of age and weighing≥40 kg).
2. Chronic AD (according to American Academy of Dermatology Consensus Criteria) that has been present for ≥1 year before the screening visit.
3. Eczema Area and Severity Index (EASI) score 16 at the baseline visit.
4. Investigator Global Assessment (IGA) score≥3 (scale of 0 to 4) at the baseline visit.
5. ≥10% body surface area (BSA) of AD involvement at the baseline visit.
6. History of inadequate response to treatment with topical medications.
7. Applied a stable dose of non-medicated topical moisturizer at least twice daily for ≥7 days prior to the baseline visit.
8. Completed electronic diary entries for pruritus and sleep-loss for a minimum of 4 of 7 days preceding randomization.
9. Willing and able to comply with all clinic visits and study-related procedures and questionnaires.
10. For women of childbearing potential: agree to remain abstinent or use a highly effective contraceptive method during the treatment period and for at least 18 weeks after the last dose of lebrikizumab or placebo.
11. Male patients must agree to use an effective barrier method of contraception during the study and for a minimum of 18 weeks following the last dose of study drug if sexually active with a female of child bearing potential.
12. Provided signed informed consent/assent.

Exclusion Criteria: Patients meeting any of the criteria below will be excluded from this study:
1. Participation in a prior lebrikizumab clinical study.
2. History of anaphylaxis as defined by the Sampson criteria (Sampson 2006).
3. Treatment with topical corticosteroids (TCS), topical calcineurin inhibitors (TCI), or Phosphodiesterase-4 inhibitors such as crisaborole within 1 week prior to the baseline visit.
4. Treatment with any of the following agents within 4 weeks prior to the baseline visit:
   a. lmmunosuppressive/immunomodulating drugs (e.g., systemic corticosteroids, cyclosporine, mycophenolate-mofetil, IFN-γ, Janus kinase inhibitors, azathioprine, methotrexate, etc.);
   b. Phototherapy and photochemotherapy (PUVA) for AD.
5. Treatment with the following prior to the baseline visit:
   a. An investigational drug within 8 weeks or within 5 half-lives (if known), whichever is longer.
   b. Dupilumab within 8 weeks.
   c. B Cell-depleting biologics, including rituximab, within 6 months.
   d. Other biologics within 5 half-lives (if known) or 16 weeks, whichever is longer.
6. Use of prescription moisturizers within 7 days of the baseline visit.
7. Regular use (more than 2 visits per week) of a tanning booth/parlor within 4 weeks of the screening visit.
8. Treatment with a live (attenuated) vaccine within 12 weeks of the baseline visit or planned during the study.
9. Uncontrolled chronic disease that might require bursts of oral corticosteroids, e.g., co-morbid severe uncontrolled asthma (defined by an ACQ-5 score≥1.5 or a history of ≥2 asthma exacerbations within the last 12 months requiring systemic [oral and/or parenteral] corticosteroid treatment or hospitalization for >24 hours).
10. Active chronic or acute infection requiring treatment with systemic antibiotics, antivirals, antiparasitics, antiprotozoals, or antifungals within 2 weeks before the baseline visit, or superficial skin infections within 1 week before the baseline visit.
11. Evidence of active acute or chronic hepatitis (as defined by the Department of Health & Human Services Centers for Disease Control and Prevention) or known liver cirrhosis.
12. Diagnosed active endoparasitic infections or at high risk of these infections.
13. Known or suspected history of immunosuppression, including history of invasive opportunistic infections (e.g., tuberculosis [TB], histoplasmosis, listeriosis, coccidioidomycosis, pneumocystosis, and aspergillosis) despite infection resolution: or unusually frequent, recurrent, or prolonged infections, per the Investigator's judgment.
14. History of human immunodeficiency virus (HIV) infection or positive HIV serology at screening.
15. In the Investigator's opinion, any clinically significant laboratory results from the chemistry, hematology or urinalysis tests obtained at the screening visit.
16. Presence of skin comorbidities that may interfere with study assessments.
17. History of malignancy, including mycosis fungoides, within 5 years before the screening visit, except completely treated in situ carcinoma of the cervix, completely treated and resolved non-metastatic squamous or basal cell carcinoma of the skin.
18. Severe concomitant illness(es) that in the Investigator's judgment would adversely affect the patient's participation in the study. Any other medical or psychological condition that in the opinion of the Investigator may suggest a new and/or insufficiently understood disease, may present an unreasonable risk to the study patient because of his/her participation in this clinical trial, may make patient's participation unreliable, or may interfere with study assessments.
19. Pregnant or breastfeeding women, or women planning to become pregnant or breastfeed during the study.
20. Have had an important side effect to TCS (e.g., intolerance to treatment, hypersensitivity reactions, significant skin atrophy, and systemic effects), as assessed by the investigator or treating physician that would prevent further use.

### Study Drug:

Lebrikizumab is provided in a sterile liquid solution containing 125 mg/mL lebrikizumab, histidine acetate, sucrose, polysorbate 20, and sterile water for injection, pH 5.4-6.0. Placebo solution is identical in appearance and content except for lebrikizumab. All study drugs (lebrikizumab and placebo) are supplied as a sterile pre-filled syringe with a pre-assembled needle safety device (PFS-NSD). Each pre-filled syringe is intended for a single 2 mL dose (250 mg) administered subcutaneously (SC).

### Results for Patients Previously Administered Dupilumab

In this study, 211 patients were randomized 2:1 to Lebrikizumab plus TCS (N=145) or placebo plus TCS (N=66). Prior exposure to dupilumab was reported in 20 patients randomized to Lebrikizumab plus TCS, and 9 patients randomized to placebo plus TCS. Of these 29 patients, the reason for prior discontinuation of dupilumab was due to loss of response or inadequate response (14 patients), intolerance to medication (1 patient), or other (14 patients). Other reported reasons for discontinuation include subject decision, affordability, treatment availability, and other.

In patients treated with Lebrikizumab plus TCS, baseline characteristics were comparable between the overall Lebrikizumab-treated population (N=145) and the subpopulation with prior dupilumab exposure (N=20). Of the 20 patients with prior dupilumab exposure, 13 patients presented with an IGA of 3, and 7 patients presented with an IGA of 4. The mean (standard deviation [SD]) body surface area reported at baseline was 37.3 (15.7). The mean (SD) baseline scores for EASI and Pruritus NRS were 25.9 (8.1) and 7.8 (1.9), respectively. The mean (SD) duration since AD onset was longer in patients with prior dupilumab experience versus those without prior dupilumab exposure (27.0 [22.5] vs 21.0 [17.4], respectively).

In patients treated with Lebrikizumab plus TCS with prior dupilumab exposure, an IGA 0/1 with 2-point improvement was achieved by 6/18 patients, EASI-75 was achieved by 13/18 patients, and Pruritus NRS 4-point improvement was achieved by 8/15 patients at Week 16. Rescue medication use with high potency or systemic medication was reported in one patient.

The results of this subpopulation analysis suggest that patients with prior dupilumab exposure are likely to respond to Lebrikizumab plus TCS. Lebrikizumab could provide clinically meaningful improvements in atopic dermatitis for patients previously administered dupilumab but failed dupilumab or those patients who are intolerant to dupilumab.

### Example 2. A Study to Evaluate the Safety and Efficacy of Lebrikizumab in Adult and Adolescent Participants with Moderate-to-Severe Atopic Dermatitis Previously Treated with Dupilumab (KGBO)

This trial is designed to evaluate the safety and efficacy of lebrikizumab in adult and adolescent participants with moderate-to-severe AD who have been previously treated/administered with dupilumab.

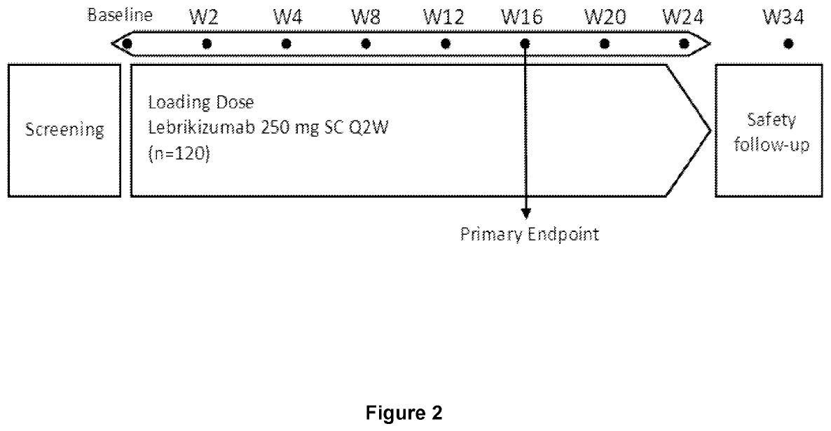

FIG. 2 is a schematic diagram of the study design. This trial has three study periods: Screening (≤30 days prior to baseline); Treatment (24 weeks); and Safety follow-up (at Week 34 or approximately 12 weeks after last treatment). During the 24-week Treatment Period, approximately 120 participants receive lebrikizumab subcutaneously (SC) at a loading dose of 500 mg at baseline (Week 0) and Week 2, followed by 250 mg once every two weeks (Q2W).

Approximately 120 participants previously treated/administered with dupilumab are enrolled in this study. Among those, approximately 80 participants are enrolled who discontinued dupilumab due to: a) no response to treatment, b) a partial response to treatment, or c) who lost response to treatment. Additionally, approximately 15 participants are enrolled who discontinued dupilumab treatment due to intolerance or adverse events (AEs). Finally, approximately 25 participants are enrolled who stopped dupilumab treatment for other reasons (for example, cost, insurance coverage, access to medication, previous trial participants). Approximately 10% (n=12) of participants are adolescents (12 to less than 18 who weigh at least 40 kg).

Table 4 lists the objectives and endpoints of this study. Statistical analyses are performed for the endpoints.

TABLE 4
Objectives and Endpoints

| Objectives | Endpoints |
|---|---|
| Primary | |
| To evaluate efficacy of lebrikizumab 250 | Percentage of participants achieving EASI-75 |
| mg Q2W on reducing signs and | at Week 16 |
| symptoms of AD at Week 16 in | |
| participants with moderate-to-severe AD | |
| previously treated with dupilumab | |
| Secondary | |
| To evaluate the efficacy of lebrikizumab | Percentage of participants achieving EASI-75 |
| 250 mg Q2W on reducing signs and | at Week 24 |
| symptoms of AD at Week 16 and Week | Percentage of participants with an IGA score of |
| 24 in participants with moderate-to- | 0 or 1 and a reduction ≥2 points from baseline |
| severe AD previously treated with | to Weeks 16 and 24 |
| dupilumab | Percentage change EASI from baseline to |
| | Weeks 16 and 24 |
| | Change in EASI from baseline to Weeks 16 |
| | and 24 |
| | Percentage of participant achieving EASI-90 |
| | from baseline to Weeks 16 and 24 |
| | Percentage of participants with a Pruritus NRS |
| | of ≥4 points at baseline who achieve at least 4- |
| | point reduction at Weeks 16 and 24 |
| | Percentage of participants with a Pruritus NRS |
| | of ≥3 points at baseline who achieve at least 3- |
| | point reduction at Weeks 16 and 24 |
| | Percentage change in Pruritus NRS score from |
| | baseline to Weeks 16 and 24 |
| | Percentage of participants with a Sleep-Loss |
| | Scale of ≥2 points at baseline who achieve at |
| | least 2-point reduction at Weeks 16 and 24 |
| | Change in Sleep-Loss Scale from baseline at |
| | Weeks 16 and 24 |
| | Percentage of participants with a Skin Pain |
| | NRS of ≥4 points at baseline who achieve a 4- |
| | point reduction from baseline at Weeks 16 and |
| | 24 |
| | Change in Skin Pain NRS from baseline to |
| | Weeks 16 and 24 |
| | Change in DLQI from baseline to Weeks 16 |
| | and 24 for participants >16 years of age at |
| | baseline |
| | Percentage of participants with at least 4 point |
| | at baseline achieving ≥4-point improvement in |
| | DLQI from baseline at Weeks 16 and 24 for |
| | participants >16 years of age at baseline |
| | Change in cDLQI from baseline to Weeks 16 |
| | and 24 for participants <16 years of age at |
| | baseline |
| | Percentage change in SCORAD from baseline |
| | to Weeks 16 and 24 |
| Exploratory | |
| To evaluate the efficacy of lebrikizumab | Percentage change from baseline in mTLss |
| 250 mg Q2W in participants with | (hands) at Weeks 16 and 24 |
| moderate-to-severe AD previously | Percentage change in ADCT from baseline by |
| treated with dupilumab | visit |
| | Proportion of TCS/TCI-free days from baseline |
| | to Weeks 16 and 24 |
| | Using WPAI-AD by visit, change in baseline in: |
| | absenteeism, in those who are currently |
| | employed (working for pay) |
| | presenteeism, in those who are currently |
| | employed (working for pay), |
| | overall work impairment, in those who are |
| | currently employed (working for pay), |
| | impairment in activities (all participants) |
| | Percentage of participants with an F-IGA score |
| | of 0 or 1 and a reduction ≥2 points from |
| | baseline by visit in those who have baseline at |
| | least 2 points |
| | Percentage of participants achieving EASI-75 |
| | by visit |
| | Percentage of participants with an IGA score of |
| | 0 or 1 and a reduction ≥2 points from baseline |
| | by visit |
| | Percentage change in EASI by visit |
| | Change in EASI by visit |
| | Percentage of participants achieving EASI-90 |
| | by visit |
| | Percentage of participants with a Pruritus NRS |
| | of ≥4 points at baseline who achieve a 4-point |
| | reduction by visit |
| | Percentage of participants with a Pruritus |
| | NRS of ≥3 points at baseline who achieve a 3- |
| | point reduction by visit |
| | Percentage change in Pruritus NRS score from |
| | baseline to Weeks 16 and 24 |
| | Percentage of participants with a Sleep-Loss |
| | Scale of ≥2 points at baseline who achieve a 2- |
| | point reduction by visit |
| | Change in Sleep-Loss Scale from baseline by |
| | visit |
| | Percentage of participants with a Skin Pain |
| | NRS of ≥4 points at baseline who achieve a 4- |
| | point reduction from baseline by visit |
| | Distribution of responses for participant |
| | satisfaction (1 question) at Weeks 16 and 24 |
| To discern responder variability within | Changes from baseline in serum proteins |
| the study and to track response | related to AD pathogenesis. |
| variability to lebrikizumab | |

### Patient Population

Inclusion Criteria: Participants are eligible to be included in the study only if all of the following criteria apply:
1. Participant must be ≥12 years of age inclusive at the time of signing the informed consent/assent. Adolescents (12-18 years of age) body weight must be ≥40 kg at baseline.
2. All participants must have prior treatment/administration with dupilumab meeting one of the following conditions:
   a. Participants who stopped dupilumab due to non-response, partial response, loss of efficacy must have been previously treated with dupilumab (at labeled dose level) for at least 4 months.
   b. Participants who stopped dupilumab due to intolerance or AEs to the drug may enter the study with no required prior length of dupilumab treatment.
   c. Participants who stopped dupilumab due to other reasons such as cost or loss of access to dupilumab (for example, insurance coverage) may enter the study with no required prior length of dupilumab treatment.
3. Participants who have chronic AD (according to American Academy of Dermatology Consensus Criteria; Eichenfield et al. 2014) that has been present for ≥1 year before screening visit.
4. Have EASI≥16 at the baseline visit.
5. Have IGA score≥3 (Scale of 0 to 4) at the baseline visit.
6. Have ≥10% body surface area (BSA) of AD involvement at the baseline visit.
7. Have a history of inadequate response to treatment with topical medications; or determination that topical treatments are otherwise medically inadvisable.
8. Female participants of child-bearing potential: must agree to remain abstinent or use a highly effective contraceptive method during the treatment period and for at least 18 weeks after the last dose of study drug. Male participants are not required to use any contraception except in compliance with specific local government study requirements.
9. Adult participants must provide signed informed consent. For adolescent participants: a parent or legal guardian must be able to read, understand, and give documented informed consent for a child to participate in this study.

Exclusion Criteria: Participants are excluded from the study if any of the following criteria apply:
1. History of human immunodeficiency virus (HIV) infection or positive HIV serology at screening.
2. Have a current infection or chronic infection with hepatitis B virus (HBV) at screening.
3. Have a current infection with hepatitis C virus (HCV) at screening.
4. Have an uncontrolled chronic disease that might require multiple intermittent uses of oral corticosteroids at screening, as defined by the investigator.
5. Have uncontrolled asthma that
   a. might require bursts of oral or systemic corticosteroids, or
   b. required the following due to ≥1 exacerbations within 12 months before baseline
      systemic (oral and/or parenteral) corticosteroid treatment, or
      hospitalization for ≥24 hours.
6. Have known liver cirrhosis and/or chronic hepatitis of any etiology.
7. History of malignancy, including mycosis fungoides or cutaneous T-cell lymphoma, within 5 years before the screening, except completely treated in situ carcinoma of the cervix of completely treated and resolved non-metastatic squamous or basal cell carcinoma of the skin with no evidence of recurrence in the past 12 weeks.
8. Are diagnosed with active endoparasitic infections or at high risk of these infections.
9. Have a known or suspected history of immunosuppression, including history of invasive opportunistic infections (for example, tuberculosis, histoplasmosis, listeriosis, coccidioidomycosis, pneumocystosis, and aspergillosis) despite infection resolution; or unusually frequent, recurrent, or prolonged infections, per the investigator's judgment.
10. Have presence of skin comorbidities that may interfere with study assessments.
11. Have a severe concomitant illness(es) that in the investigator's judgment would adversely affect the participant's participation in the study.
12. Have any other medical or psychological condition that in the opinion of the investigator may suggest a new and/or insufficiently understood disease, may present an unreasonable risk to the study participant because of their participation in this clinical trial, may make participant's participation unreliable, or may interfere with study assessments.
13. Have had any of the following types of infection within 3 months of screening or develop any of these infections during screening:
   a. Serious (requiring hospitalization, and/or intravenous or equivalent oral antibiotic treatment);
   b. Opportunistic (as defined in Winthrop et al. 2015,);
   c. Chronic (duration of symptoms, signs, and/or treatment of 6 weeks or longer);
   d. Recurring (including, but not limited to recurring cellulitis, chronic osteomyelitis).
14. Have an active or acute infection requiring treatment with systemic antibiotics, antivirals, antiparasitics, antiprotozoals, or antifungals within 2 weeks before the baseline visit (baseline), or superficial skin infections within 1 week before the baseline visit.
15. Dupilumab treatment within 4 weeks prior to the baseline visit.
16. Prior treatment with tralokinumab.
17. Treatment with topical agents (corticosteroids, calcineurin inhibitors, JAK inhibitors, or phosphodiesterase-4 inhibitors) within 2 weeks prior to baseline visit.
18. Treatment with any of the following agents within 4 weeks prior to the baseline visit:
19. systemic immunosuppressive/immunomodulating drugs (for example, systemic corticosteroids, cyclosporine, mycophenolate mofetil, IFN-gamma, azathioprine, methotrexate, and other immunosuppressants);
20. small molecules (for example, JAK inhibitors);
21. phototherapy and photochemotherapy for AD.
22. Have regularly used (more than 2 visits per week) a tanning booth or parlor within 4 weeks of the screening visit (Visit 1).
23. Are currently receiving build-up dosing of allergen immunotherapy (allergy shots).
24. Treatment with B cell-depleting biologics, including rituximab, within 6 months prior to the baseline visit.
25. Use of prescription moisturizers within 7 days of the baseline visit.
26. Have received a Bacillus Calmette-Guerin vaccination or treatment within less than 4 weeks before the baseline visit or intend to receive Bacillus Calmette-Guerin vaccination or treatment during the study.
27. Have received any live attenuated vaccine within less than 4 weeks of the baseline visit or intend to receive a live attenuated vaccine during the study, or within 4 weeks after receiving the last dose of study intervention.
28. NOTE: The following are not considered live vaccines: RNA vaccines, vaccines with inactive viral elements, and/or non-replicating viral vector vaccines.
29. Use of cannabis or cannabinoids for the treatment of pruritus, pain and/or AD.
30. In the opinion of the investigator, have clinically significant laboratory results from the chemistry or hematology tests obtained at the screening visit.
31. Are currently enrolled in any other clinical study involving an investigational product or any other type of medical research judged not to be scientifically or medically compatible with this study.
32. Treatment with an investigational drug within 8 weeks or within 5 half-lives (if known), whichever is longer prior to the baseline visit.
33. Have received a dose of lebrikizumab in any prior lebrikizumab clinical study.

### Study Drug:

Pharmaceutical compositions containing 2 mL of 125 mg/mL lebrikizumab or placebo are supplied as sterile pre-filled syringes with needle safety device (PFS-NSD) for subcutaneous administration to the patients. Lebrikizumab sequences are provided in Table 1. The placebo solution is identical in appearance and volume to the active solution except that it does not contain lebrikizumab.

1. A method for treating moderate to severe atopic dermatitis, the method comprising:
selecting a patient who has moderate to severe atopic dermatitis and has been previously administered dupilumab and stopped dupilumab due to non-response, partial response, or loss of response, and
administering to the patient an antibody that binds human IL-13,
wherein the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

2. A method for treating moderate to severe atopic dermatitis, the method comprising:
selecting a patient who has moderate to severe atopic dermatitis and has been previously administered dupilumab and stopped dupilumab due to intolerance or adverse event, and
administering to the patient an antibody that binds human IL-13,
wherein the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

3. The method of claim 1, wherein the patient has moderate to severe atopic dermatitis for at least a year.

4. The method of claim 1, wherein the moderate to severe atopic dermatitis is determined by the American Academy of Dermatology Consensus Criteria.

5. The method of claim 1, wherein the patient has an Eczema Area and Severity Index (EASI) score of 16 or greater, an Investigator Global Assessment (IGA) score of 3 or greater, and more than 10% of body surface area (BSA) affected by atopic dermatitis, before administration of the antibody that binds human IL-13.

6. The method of claim 1, wherein the patient had inadequate response to topical corticosteroids.

7. The method of claim 1, wherein the patient is 12 years of age or older.

8. A method for treating moderate to severe atopic dermatitis, the method comprising:
selecting a patient who:
i. is 12 years of age or older;
ii. has chronic atopic dermatitis according to American Academy of Dermatology Consensus Criteria for more than a year;
iii. has an EASI score of 16 or greater;
iv. has an IGA score of 3 or greater;
v. has more than 10% of body surface area affected by atopic dermatitis;
vi. had inadequate response to topical corticosteroids; and
vii. has been previously administered dupilumab, wherein the patient stopped dupilumab due to (1) non-response, partial response, or loss of response, or (2) intolerance or adverse event; and
administering to the patient an antibody that binds human IL-13,
wherein the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

9. The method of claim 8, wherein the patient stopped dupilumab due to non-response, partial response, or loss of response.

10. The method of claim 8, wherein the patient stopped dupilumab due to intolerance or adverse event.

11. The method of claim 1, wherein the patient stopped dupilumab due to non-response.

12. The method of claim 11, wherein the patient's skin and/or itch is not improved at all or only improved less than 25% at peak response to dupilumab.

13. The method of claim 1, wherein the patient stopped dupilumab due to partial response.

14. The method of claim 13, wherein the patient's skin and/or itch is only improved between 25-50% at peak response to dupilumab.

15. The method of claim 1, wherein the patient stopped dupilumab due to loss of response.

16. The method of claim 15, wherein the patient's skin and/or itch is initially improved after dupilumab treatment, but then it worsened.

17. The method of claim 1, wherein the patient stopped dupilumab for at least four weeks.

18. The method of claim 1, wherein the patient stopped dupilumab for at least eight weeks.

19. The method of claim 1, wherein the antibody that binds human IL-13 comprises a VH comprising SEQ ID NO: 7, and a VL comprising SEQ ID NO: 8.

20. The method of claim 1, wherein the antibody that binds human IL-13 comprises a heavy chain comprising SEQ ID NO: 9, and a light chain comprising SEQ ID NO: 10.

21. The method of claim 1, wherein the antibody that binds human IL-13 is lebrikizumab.

22. The method of claim 1, wherein the antibody that binds human IL-13 is administered subcutaneously to the patient.

23. The method of claim 1, wherein the antibody that binds human IL-13 is administered at 250 mg or 500 mg subcutaneously to the patient.

24. The method of claim 1, wherein the antibody that binds human IL-13 is administered subcutaneously to the patient once every two weeks.

25. The method of claim 1, wherein the patient is treated with the antibody that binds human IL-13 for a treatment period of 16-24 weeks.

26. The method of claim 1, wherein the patient is treated with the antibody that binds human IL-13 for a treatment period of 16 weeks.

27. The method of claim 1, wherein the patient is treated with the antibody that binds human IL-13 for a treatment period of 24 weeks.

28. The method of claim 1, wherein the patient is treated with a loading dose of 500 mg of the antibody that binds human IL-13 at week 0 and week 2, and a subsequent dose of 250 mg of the antibody that binds human IL-13 once every two weeks for the remainder of treatment period.

29. The method of claim 1, further comprising determining the EASI score of the patient after the treatment period.

30. The method of claim 29, wherein the EASI score determined after the treatment period is reduced by 75% or greater compared to the EASI score determined prior to administration of the first loading dose of the antibody that binds human IL-13.

31. The method of claim 29, wherein the EASI score determined after the treatment period is reduced by 90% or greater compared to the EASI score determined prior to administration of the first loading dose of the antibody that binds human IL-13.

32. The method of claim 1, further comprising determining the IGA score of the patient after the treatment period.

33. The method of claim 32, wherein the IGA score determined after the treatment period is 0 or 1 and the IGA score determined after the treatment period is reduced by 2 points or greater compared to the IGA score determined prior to administration of the first loading dose of the antibody.

34. The method of claim 1, further comprising determining the Pruritus Numeric Rating Scale (NRS) score of the patient after the treatment period.

35. The method of claim 34, wherein the pruritus NRS score determined after the treatment period is reduced by 4 points or greater compared to the pruritus NRS score determined prior to administration of the first loading dose of the antibody that binds human IL-13.

36. The method of claim 1, further comprising determining one or more of the following characteristics of the patient after the treatment period:
i. Sleep Loss scale;
ii. Skin Pain NRS score;
iii. Severity Scoring of Atopic Dermatitis (SCORAD);
iv. Dermatology Life Quality Index (DLQI) or Children Dermatology Life Quality Index (CDLQI) score
v. Face-Investigator's Global Assessment (F-IGA);
vi. Modified Total Lesion Symptom Scale;
vii. Atopic Dermatitis Control Tool (ADCT);
viii. Participant-Reported Satisfaction Question;
ix. Fitzpatrick Skin Phototype Assessment Scale;
x. Work Productivity and Activity Impairment Questionnaire-Atopic Dermatitis (WPAI-AD);
xi. Patient-Oriented Eczema Measure (POEM);
xii. European Quality of Life-5 Dimensions-5 Levels (EQ-5D-5L);
xiii. PROMIS (Patient-Reported Outcomes Measurement Information System) Anxiety and Depression measures.

37. The method of claim 25, wherein the patient is further treated for a maintenance period up to 36 weeks.

38. The method of claim 37, wherein the patient is treated with a maintenance dose of 250 mg of the antibody that binds human IL-13 once every two weeks during the maintenance period.

39. The method of claim 37, wherein the patient is treated with a maintenance dose of 250 mg of the antibody that binds human IL-13 once every four weeks during the maintenance period.

40. The method of claim 37, wherein the patient is treated with a maintenance dose of 250 mg of the antibody that binds human IL-13 once every eight weeks during the maintenance period.

41. The method of claim 1, wherein the antibody that binds human IL-13 is administered to the patient using a subcutaneous administration device.

42. The method of claim 41, wherein the subcutaneous administration device is selected from a prefilled syringe, disposable pen injection device, microneedle device, microinfuser device, needle-free injection device, or autoinjector device.

43. The method of claim 1, wherein the method further comprises administrating one or more topical corticosteroids to the patient.

44. The method of claim 43, wherein the one or more topical corticosteroids is triamcinolone acetonide, hydrocortisone, or a combination of triamcinolone acetonide and hydrocortisone.

45. The method of claim 43, wherein the one or more topical corticosteroids is administered concomitantly with the antibody that binds human IL-13.

46. An antibody that binds human IL-13 for use in the treatment of moderate to severe atopic dermatitis in a patient who has been previously administered dupilumab and stopped dupilumab due to non-response, partial response, or loss of response, wherein the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

47. An antibody that binds human IL-13 for use in the treatment of moderate to severe atopic dermatitis in a patient who has been previously administered dupilumab and stopped dupilumab due to intolerance or adverse event, wherein the antibody that binds human IL-13 comprises a heavy chain variable region (VH) and a light chain variable region (VL), wherein the VH comprises a HCDR1 comprising SEQ ID NO: 1, a HCDR2 comprising SEQ ID NO: 2, and a HCDR3 comprising SEQ ID NO: 3, and the VL comprises a LCDR1 comprising SEQ ID NO: 4, a LCDR2 comprising SEQ ID NO: 5, and a LCDR3 comprising SEQ ID NO: 6.

48. The antibody for use of claim 46, wherein the patient:
i. is 12 years of age or older;
ii. has chronic atopic dermatitis according to American Academy of Dermatology Consensus Criteria for more than a year;
iii. has an EASI score of 16 or greater;
iv. has an IGA score of 3 or greater;
v. has more than 10% of BSA affected by atopic dermatitis; and
vi. had inadequate response to topical corticosteroids.

49.-53. (canceled)
